(19) United States
(12) Patent Application Publication
(10) Pub. No.: US 2011/0243950 A1
(43) Pub. Date: Oct. 6, 2011

(54) MSP2 ANTIGENIC PEPTIDES AND THEIR USE

(76) Inventors: Giampietro Corradin, Lausanne (CH); Ingrid Felger, Allschwil (CH)

(21) Appl. No.: 13/132,843

(22) PCT Filed: Dec. 3, 2009
(86) PCT No.: PCT/IB2009/055487

§ 371 Date: Jun. 3, 2011

Related U.S. Application Data
(60) Provisional application No. 61/121,060, filed on Dec. 9, 2008.
(60) Provisional application No. 61/120,147, filed on Dec. 5, 2008.

(51) Int. Cl. A61K 39/395; C07K 14/445; C07K 7/06; A61K 39/015; C07K 16/20; C07H 21/00; G01N 33/566; C12N 5/16; C12N 15/63; A61P 37/04; A61P 33/02; A61P 33/06

(52) U.S. Cl. 424/139.1; 530/350; 530/324; 530/328; 424/191.1; 530/387.9; 536/23.5; 436/501; 435/331; 435/320.1

(57) ABSTRACT

This invention relates generally to the field of P. falciparum antigens and their use, for example, for the preparation of a vaccine against said pathogen. More specifically, the present invention relates to an antigenic peptide derived from the constant part of MSA2, and includes antibodies and methods of producing and using same.

### FIELD OF THE INVENTION

This invention relates generally to the field of pathogen peptidic antigens and their use, for example, for the preparation of a vaccine against said pathogen. More specifically, the present invention relates to an antigenic peptide derived from the constant part of Plasmodium falciparum sequences, and includes antibodies and methods of producing and using same.

### BACKGROUND OF THE INVENTION

Plasmodium falciparum, the causative agent of the most severe form of malaria infects 500 million people per year and kills at least one million. One of the most cost-effective intervention to curb the disease is the development of an effective vaccine, which to date is not available. The reasons for this are mainly due to the complex life cycle of the parasite, its antigenic variation and diversity, the wide variety of immune responses it induces, and the incomplete knowledge of protective immunity mechanisms. Individuals living in malaria endemic regions develop a clinical immunity associated with high antibody titres against major surface molecules of the merozoite stage, however, immunity is never sterile. Passive transfer studies have shown that immunoglobulin from semi-immune individuals can confer clinical immunity to individuals exposed to geographically diverse parasite strains (McGregor et al.), (Sabchareon et al.). Vaccines against the blood stages of the parasite could accelerate the acquisition of natural immunity. They do not aim at preventing infection, but at protecting from morbidity and mortality. An advantage of this type of vaccine is constant boosting of the immune response by naturally occurring infections.

The polymorphic merozoite surface protein 2 (MSP2) of Plasmodium falciparum is considered a vaccine candidate because several studies have shown that high antibody titres to MSP2 are associated with protection against P. falciparum malaria (Metzger et al.). MSP2-specific antibodies in immune individuals are found to be predominantly of the cytophilic IgG3 subclass (Taylor et al.; Metzger et al.). MSP2 consists of a highly polymorphic central repeat region flanked by a dimorphic region that defines the two allelic families of MSP2, 3D7 and FC27, respectively. The N- and C-terminal domains are completely conserved. The function of MSP2 remains unclear but appears to be essential, because targeted gene disruption failed to produce viable parasites (Cowman et al.). The location of MSP2 on the merozoite surface suggests a role in invasion, and monoclonal antibodies to MSP2 inhibited the invasion of merozoites into erythrocytes (Epping et al.). Natural antibody responses have been shown to be directed mainly against the dimorphic and polymorphic regions but not against the highly conserved termini (Thomas et al.; Metzger et al.). Peptides corresponding to polymorphic and conserved parts of the molecule were immunogenic in mice (Saul et al.).

Combination B, which contained full-length 3D7 MSP2, block 2, 3 of MSP1, and ring-infected erythrocyte surface antigen (RESA) showed a promising efficacy against parasite density (62%) when tested in a phase I-IIb field trial in Papua New Guinean children (Genton et al.). The activity of the MSP2 subunit against parasite density was suggested by a selective effect of the vaccine in favour of parasite strains carrying an MSP2 allele belonging to the FC27 allelic family not presented in the vaccine (Genton et al.). However, the MSP2 construct used in Combination B included the polymorphic repeat region, which has been found to be immunogenic but cannot protect against a wide range of parasite strains.

Although there has been some progress in the treatment of malaria, the development of a safe and effective malaria vaccine remains an urgent unmet medical need for vast populations living in malaria-endemic region.

This object has been achieved by providing a new antigenic peptide derived from the constant part of polymorphic merozoite surface protein 2 (MSP2) of Plasmodium falciparum.

### SUMMARY OF THE INVENTION

The present invention provides a new antigenic peptide derived from the constant part of at least one of the two allelic families of the merozoite surface protein 2 (MSP2) of Plasmodium falciparum, biologically active fragments thereof, molecular chimeras thereof, combinations thereof and/or variants thereof.

Another object of the invention is to provide an antigenic cocktail composition derived from the constant part of at least one of the two allelic families of the merozoite surface protein 2 (MSP2) of Plasmodium falciparum.

This invention also contemplates the use of the antigenic peptide derived from the constant part of at least one of the two allelic families of the merozoite surface protein 2 (MSP2) of Plasmodium falciparum or of the antigenic cocktail composition in the preparation of a vaccine composition useful to stimulate an immune response in a mammal and the use thereof in the manufacture of a medicament for the treatment and/or prevention of malaria

A further object of the present invention is to provide an antibody that recognizes the antigenic peptide or the antigenic cocktail composition derived from the constant part of at least one of the two allelic families of the merozoite surface protein 2 (MSP2) of Plasmodium falciparum.

The present invention also relates to a purified and isolated nucleic acid sequence comprising a nucleotidic sequence encoding the antigenic peptide of the invention, an expression vector comprising at least one copy of said purified and isolated nucleic acid sequence, and a host cell comprising either the purified and isolated nucleic acid sequence or the expression vector.

A diagnostic tool for determining the presence of the antigenic peptide of the invention or antibodies directed against said antigenic peptide is also contemplated in the present invention

Other objects and advantages will become apparent to those skilled in the art from a review of the ensuing detailed description, which proceeds with reference to the following illustrative drawings, and the attendant claims.

### DESCRIPTION OF THE FIGURES

FIG. 1: Amino acid sequence alignment of full-length MSP2 alleles and long synthetic peptides.

Peptides MR141 (3D7 family-specific part+conserved C-terminal part) and MR144 A (FC27 family-specific part+conserved C-terminal part) were used for affinity-purification of antibody from human immune sera. Peptide MR141 and MR144A were also used for antigenicity and immunogenicity studies in mice, for production of monoclonal antibodies as well as for the analysis of association with protection in children. Non-repetitive family-specific parts are shaded in light gray, conserved sequences are shaded in dark gray. The bold and underlined asparagine residue (N) represents the putative cleavage site for the addition of the glycosylphosphatidylinositol (GPI) anchor. The two cysteine residues in the C-terminus (marked with asterisks) were either reduced or oxidized in the peptides used for affinity purification, antigenicity and immunogenicity studies.

FIG. 2: Recognition of long synthetic MSP2 peptides by Papua New Guinean immune sera. Enzyme-linked immuno sorbent assays (ELISA) on synthetic MSP2 peptides were used to determine the antibody titers of adult sera from a region in Papua New Guinea where malaria is highly endemic. Ninety-six % of the tested sera recognised peptide MR141 (3D7-cons), 93% recognised peptide MR144 A (FC27-cons), and 43% recognised peptide MR140 (cons). The median OD value was 0.84 for the 3D7-cons peptide with 50% of the values lying between OD 0.31 and 1.83. The median OD value for FC27-cons was 0.42 (50% percentile from 0.09 to 0.49) and 0.16 (0.11 to 0.27) for the C-terminal conserved peptide (cons).

FIG. 3: immunofluorescence microscopy analysis of P. falciparum parasites with MSP2-peptide specific antibodies.

Acetone/methanol-fixed P. falciparum schizonts and merozoites were reacted with MSP2-specific antibodies. (A) Human antibody affinity-purified on peptide MR141 (3D7cons) on 3D7 parasites. (B) Human antibody affinity-purified on peptide MR144 A (FC27cons) on K1 parasites. (C) Mouse monoclonal antibody raised against peptide MR141 with an epitope mapped to the 3D7-specific region (C) and to the C-terminal conserved region (D) on 3D7 parasites. Left hand panels show parasite nuclei stained with DAPI, central panels show the MSP2 antibody labelling followed by a Cy3-conjugated anti-human IgG-specific (A, B) or anti-mouse IgG-specific antibody (C, D), respectively. The right hand panels are a merge of the blue and red fluorescence channels.

FIG. 4: IgG3 is the dominant subclass in peptide-purified MSP2-specific antibodies from human immune sera. ELISA showing the IgG subclass distribution in MSP2-specific antibody affinity-purified from human immune sera on peptide MR141 (A) and peptide MR144 A (B). Microtitre plates were coated with 1 μg/ml of the corresponding peptide and reacted with serial dilutions of affinity-purified human antibodies followed by IgG subclass-specific anti-human antibodies. OD values are given for each IgG subclass: IgG1 (diamonds), IgG2 (open squares), IgG3 (closed triangles), and IgG4 (X). Values represent mean values of an experiment run in duplicate.

FIG. 5: Specificity and cross-reactivity of purified human antibodies. ELISA showing the reactivity of peptide-purified human antibodies anti-MR141, anti-MR144A, and a control antibody anti-MR127 (purified from the same serum pools) on peptides MR141, MR144A, and MR140 (represented by diamonds, squares, and triangles, respectively). OD values are given for four different antibody dilutions.

### DETAILED DESCRIPTION OF THE INVENTION

The present invention relates to an antigenic peptide derived from the constant part of at least one of the two allelic families of the merozoite surface protein 2 (MSP2) of Plasmodium falciparum.

The merozoite surface protein 2 (MSP2) of Plasmodium falciparum consists of a highly polymorphic central repeat region flanked by a dimorphic region that defines two allelic families of MSP2, 3D7 and FC27, respectively. The N- and C-terminal domains are completely conserved. The function of MSP2 remains unclear but appears to be essential, because targeting gene disruption failed to produce viable parasites. The location of MSP2 on the merozoite surface suggested a role in invasion, and monoclonal antibodies to MSP2 have been shown to inhibit the invasion of merozoites into erythrocytes. Natural antibody responses have been shown to be directed mainly against the dimorphic and polymorphic regions but not against the highly conserved termini.

The term “comprising” is generally used in the sense of including, that is to say permitting the presence of one or more features or components.

As used herein, the terms “protein”, “polypeptide”, “polypeptidic”, “peptide” and “peptidic” are used interchangeably herein to designate a series of amino acid residues connected to the other by peptide bonds between the alpha-amino and carboxy groups of adjacent residues.

The term “antigenic peptide” refers to a peptide that is recognized by an antibody. Usually, the immune response results in the production of antibodies recognizing the antigenic peptide or at least a part or fragment thereof.

In the present context, the term “derived” refers to the fact that the antigenic peptide has been selected among the amino acid sequences of the merozoite surface protein 2 (MSP2) of Plasmodium falciparum. More particularly, the amino acid sequences of the merozoite surface protein 2 (MSP2) are chosen in the C-terminal conserved part of the 3D7 protein or in the C-terminal conserved part of the FC27 protein of Plasmodium falciparum

“Plasmodium falciparum” refers to a protozoan parasite, one of the species of Plasmodium that cause malaria in humans. P. falciparum comprises the following strains: Plasmodium falciparum (isolate 311), Plasmodium falciparum (isolate 7G8), Plasmodium falciparum (isolate CAMP/Malaysia), Plasmodium falciparum (isolate CDC/Honduras), Plasmodium falciparum (isolate DD2), Plasmodium falciparum (isolate FC27/Papua New Guinea), Plasmodium falciparum (isolate FcB1/Columbia), Plasmodium falciparum (isolate FCBR/Columbia), Plasmodium falciparum (isolate FCH-5), Plasmodium falciparum (isolate FCM17/Senegal), Plasmodium falciparum (isolate FCR-3/Gambia), Plasmodium falciparum (isolate fid3/india), Plasmodium falciparum (isolate hb3), Plasmodium falciparum (isolate IMR143), Plasmodium falciparum (isolate K/Thailand), Plasmodium falciparum (isolate KF1916), Plasmodium falciparum (isolate LE5), Plasmodium falciparum (isolate mad20/papua new guinea), Plasmodium falciparum (isolate mad71/papua new guinea), Plasmodium falciparum (isolate NF54), Plasmodium falciparum (isolate NF7/Ghana), Plasmodium falciparum(isolate nig32/nigeria), Plasmodium falciparum (isolate PALO ALTO/UGANDA), Plasmodium falciparum (isolate RO-33/Ghana), Plasmodium falciparum (isolate T4/Thailand), Plasmodium falciparum (isolate TAK 9), Plasmodium falciparum (isolate thtn/thailand), Plasmodium falciparum (isolate V1), Plasmodium falciparum (isolate WELLCOME), and Plasmodium falciparum 3D7.

Usually, the antigenic peptide derived from the constant part of at least one of the two allelic families of the merozoite surface protein 2 (MSP2) of Plasmodium falciparum will be selected from the group comprising the amino acid sequences SEQ ID No 1 (MR141): AEASTSTSSENPNHKNAETNPKGKGEVQEPNQANKETQNNSNVQQDSQTKSNVPPTQDADTKSPTAQP EQAENSAPTAEQTESPELQSAPENKGTGQHGHMHGSRNNHPQNTSDSQKECTDGNKENCG SEQ ID No 2 (LR186): AEASTSTSSENPNHKNAETNPKGKGEVQEPNQANKETQNNSNVQQDSQTKSNVPPTQDADTKSPTAQP EQAENSAPTAEQTESPELQSAPENKGTG, SEQ ID No 3 (MR144A): ESSSSGNAPNKTDGKGEESEKQNELNESTEEGPKAPQEPQTAENENPAAPENKGTGQHGHMHGSRNNH PQNTSDSQKECTDGNKENCG,

biologically active fragments thereof, molecular chimeras thereof, combinations thereof and/or variants thereof.

“Biologically active fragments” refer to a part of a sequence containing less amino acids in length than the sequence of the peptide of the invention. This sequence can be used as long as it exhibits similar immunogenic properties as the native sequence from which it derives and provided that said biologically active fragment has at least one of the following properties:

i) said biologically active fragment has retained the property of inducing, at least one of IgG1, IgG2, IgG2a, IgG2b, IgG3 IgG4 antibodies, more particularly, specific IgG1 and/or IgG3;

ii) said biologically active fragment has retained the property of inducing antibodies that are specific of the Plasmodium-infected erythrocytes;

iii) in the ADCI assay, said biologically active fragment has retained its property of inducing specific antibodies that have an inhibitory effect on Plasmodium growth that is of at least 30%, e.g., it has retained the property of inducing specific IgG1 and/or IgG3, wherein said induced IgG1 and/or IgG3 have a SGI value of at least 30%, preferably of at least 70%, even more preferably of at least 80% in the ADCI assay;

iv) said biologically active fragment has retained the property that, in human beings under natural exposure to the parasite, it induces specific antibodies (IgG1 and/or IgG3) that are very strongly associated with a state of resistance to malaria;

v) said biologically active fragment has retained the property that parasite-induced antibodies, which are specific of said biologically active fragment, are present in individuals, who resist to malaria and are absent, or are present at lower titres, in individuals, who have malaria attack.

Preferably this sequence contains less than 90%, preferably less than 60%, in particular less than 30% amino acids in length than the respective sequence of the peptide of the invention. Preferably also these sequences contain at least 8, most preferably 25, more preferably 40, even more preferably 50 and still even more preferably 88 contiguous amino acids in length in common with sequence of the peptide of the invention.

Preferably, these biologically active fragments will be selected from the group comprising sequences:

SEQ ID N^{o} 4:
ESSSSGNAPNKTDGKGEESEKQNELNESTEEGPKAPQEPQTAENENPA,
SEQ ID N^{o} 5 (MR140):
APENKGTGQHGHMHGSRNNHPQNTSDSQKECTDGNKENCG,
SEQ ID N^{o} 6:
AEASTSTSSENPNHKNAETNPKGKGEVQEPNQANKETQNNSNVQQDSQTK
SNVPPTQDADTKSPTAQPEQAENSAPTAEQTESPELQS,
SEQ ID N^{o} 7:
APENKGTG.

The inventors of the present invention have shown that the antigenic peptide derived from the constant part of from the merozoite surface protein 2 (MSP2) of Plasmodium falciparum, biologically active fragments thereof, molecular chimeras thereof, combinations thereof and/or variants thereof have a high antigenicity as well as a high immunogenicity.

The antigenic peptide and biologically active fragments prepared by a variety of methods and techniques known in the art such as for example chemical synthesis (e.g. single or multi-channel peptide synthesizer).

Furthermore, since an inherent problem with native peptides (in L-form) is the degradation by natural proteases, the peptide of the invention may be prepared in order to include D-forms and/or “retro-inverso isomers” of the peptide. Preferably, retro-inverso isomers of short parts, variants or combinations of the peptide of the invention are prepared.

By “retro-inverso isomer” is meant an isomer of a linear peptide in which the direction of the sequence is reversed and the chirality of each amino acid residue is inverted; thus, there can be no end-group complementarity.

Protecting the peptide from natural proteolysis should therefore increase the effectiveness of the specific heterobivalent or heteromultivalent compound. A higher biological activity is predicted for the retro-inverso containing peptide when compared to the non-retro-inverso containing analog owing to protection from degradation by native proteinases. Furthermore they have been shown to exhibit an increased stability and lower immunogenicity (Sela M. and Zisman E.).

Retro-inverso peptides are prepared for peptides of known sequence as described for example in Sela and Zisman.

Also encompassed by the present invention are modifications of the antigenic peptide (which do not normally alter primary sequence), including in vivo or in vitro chemical derivitization of peptides, e.g., acetylation or carboxylation. Also included are modifications of glycosylation, e.g., those made by modifying the glycosylation patterns of a peptide during its synthesis and processing or in further processing steps, e.g., by exposing the peptide to enzymes which affect glycosylation e.g., mammalian glycosylating or deglycosylating enzymes. Also included are sequences which have phosphorylated amino acid residues, e.g., phosphotyrosine, phosphoserine, or phosphothreonine. Additionally, cysteine residues are either in a reduced or oxidized form.

Encompassed by the present invention is also a molecular chimera of the antigenic peptide of the invention. By “molecular chimera” is intended a polynucleotide or polypeptide sequence that may include a functional portion of the antigenic peptide and that will be obtained, for example, by protein chemistry techniques known by those skilled in the art.

Particular combinations of the antigenic peptide sequence or fragments or subportions thereof are also considered in the present invention. Preferably, such combination or antigenic cocktail is obtained by combining fragments from the 3D7 and FC-27 allelic families.

The present invention also includes variants of the antigenic peptide. The term “variants” refer to polypeptides having amino acid sequences that differ to some extent from a native sequence polypeptide, that is amino acid sequences that vary from the native 3D sequence whereby one or more amino acids are substituted by another one. The variants can occur naturally (e.g. polymorphism) or can be synthesized. Variants possess substitutions, deletions, and/or insertions at certain positions within the amino acid sequence of the native amino acid sequence. Amino acid substitutions are herein defined as exchanges within one of the following five groups:

I. Small aliphatic, nonpolar or slightly polar residues: Ala, Ser, Thr, Pro, Gly
II. Polar, positively charged residues: His, Arg, Lys
III. Polar, negatively charged residues: and their amides: Asp, Asn, Glu, Gln
IV. Large, aromatic residues: Phe, Tyr, Trp
V. Large, aliphatic, nonpolar residues: Met, Leu, Ile, Val, Cys.
Non-natural amino acids can also be introduced by chemical synthesis.

The present invention also relates to an antigenic cocktail composition of Plasmodium species that comprises at least 2 antigenic peptides of the invention or mixtures thereof. Applicants have shown that the use of an antigenic cocktail composition of the invention improves the performance of vaccines since simultaneous immune responses to antigens, which are involved in the same or in different pathogenic mechanisms, surprisingly confers greater and sustained protection. The antigenic peptides may be free or linked together. In case said antigenic peptides are linked, they are preferably linked by a peptide bond or by the way of a linker such as PEG (Poly ethylene glycol).

Preferentially, the antigenic cocktail composition is a combination of two peptides deriving from MSP2, one belonging to the FC27 allelic family and the other to the 3D7 allelic family. Most preferably, the antigenic cocktail composition is selected from the group comprising a combination with SEQ IDs No 1 and 3 or SEQ IDs No 2 and 3.

Also encompassed by the present invention is the use of an antigenic peptide or of the antigenic cocktail composition of the invention for the preparation of a vaccine composition useful to stimulate an immune response in a mammal. “Mammal” refers to any animal classified as a mammal including humans, domestic and farm animals, and zoo, sports or pet animals, such as dogs, horses, cats, cows, monkeys, etc. Preferably the mammal is a human.

To augment the immune response elicited, it may be preferable to couple the peptides of the invention, or the antigenic cocktail composition, to any carrier molecule or carrier proteins. Various protein, glycoprotein, carbohydrate or sub-unit carriers can be used, including but not limited to, tetanus toxoid/toxin, diphtheria toxoid/toxin, pseudomonas mutant carrier, bacteria outer membrane proteins, crystalline bacterial cell surface layers, various endo or exotoxins, serum albumin, gamma globulin or keyhole limpet hemocyanin, recombinant, exotoxin A, LT toxin, Cholera B toxin, Klebsiella pneumoniae OmpA, Bacterial flagella, Clostridium difficile recombinant toxin A, Peptide dendrimers (multiple antigenic peptides), pan DR epitope (PADRE), universal T-cell epitopes from tetanus toxin, Commensal bacteria, Phage (displaying peptide on and bacteria phages), attachment of peptides to recombinant IgG1 and/or other suitable constituents like virosomes (virus like particles).

In addition, the peptides of the invention, or the antigenic cocktail composition, or their conjugates with carrier proteins may be further mixed with adjuvants to elicit an immune response, as adjuvants may increase immunoprotective antibody titers or cell mediated immunity response. Such adjuvants can include, but are not limited to, MPL+TDM+CWS (SIGMA), MF59 (an oil-in-water emulsion that includes 5% squalene, 0.5% sorbitan monoleate and 0.5% sorbitan trioleate Chiron), Heat-labile toxin (HLT), CRMig (nontoxic genetic mutant of diphtheria toxin), Squalene (IDEC PHARMACEUTICALS CORP.), Ovalbumin (SIGMA), Quil A (SARGEANT, INC.), Aluminum phosphate gel (SUPERFOS BIOSECTOR), Cholera holotoxin (CT LIST BIOLOGICAL LAB.), Cholera toxin B subunit (CTB), Cholera toxin A subunit-Protein A D-fragment fusion protein, Muramyl dipeptide (MDP), Adjumera (polyphosphazene, VIRUS RESEARCH INSTITUTE), Montanide ISA 720, SPT (an emulsion of 5% squalene, 0.2% Tween 80, 1.25% Pluronic L121 with phosphate-buffered saline ph 7. 4), Avridine (M6 PHARMACEUTICALS), Bay R1005 (BAYER), Calcitrol (SIGMA), Calcium phosphate gel (SARGEANT INC.), CRL 1005 (Block co-polymer P1205, VAXCEL CORP.), DHEA (MERCK), DMPC (GENZYME PHARMACEUTICALS and FINE CHEMICALS). DMPG (GENZYME PHARMACEUTICALS and FINE CHEMICALS), Gamma Inulin, Gerbu Adjuvant (CC BIOTECH CORP.), GM-CSF, (IMMUNE CORP.), GMDP (PEPTECH LIMITED), Imiquimod (3M PHARMACEUTICALS), ImmTher (ENDOREX CORPORATION), ISCOMTM (ISCOTEC AB), Iscoprep 7.0. 3 ™ (ISCOTEC AB), Loxoribine, LT-Oral Adjuvant (E. coli labile enterotoxin, protoxin, BERNA PRODUCTS CORP.), MTP-PE (CIBA-GEIGY LTD), Murametide, (VACSYN S. A.), Murapalmitine (VACSYN S. A.), Pluronic L121 (IDEC PHARMACEUTICALS CORP.), PMMA (INSTITUT FUR PHARMAZEUTISCHE TECHNOLOGIE), SAF-1 (SYNTEX ADJUVANT FORMULATION CHIRON), Stearyl tyrosine (BIOCHEM THERAPEUTIC INC.), Theramidea (IMMUNO THERAPEUTICS INC.), Threonyl-MDP(CHIRON), FREUNDS complete adjuvant, FREUNDS incomplete adjuvant, aluminum hydroxide, dimethyldioctadecyl-ammonium bromide, Adjuvax (ALPHA-BETA TECHNOLOGY), Inject Alum (PIERCE), Monophosphoryl Lipid A (RIBI IMMUNOCHEM RESEARCH), MPL+TDM (RIBI IMMUNOCHEM RESEARCH), Titermax (CYTRX), QS21, t Ribi Adjuvant System, TiterMaxGold, QS21, Adjumer, Calcitrol, CTB, LT (E. coli toxin), LPS (lipopolysaccharide), Avridine, the CpG sequences (Singh et al., 1999 Singh, M. and Hagum, D., Nature Biotechnology 1999 17: 1075-81) toxins, toxoids, glycoproteins, lipids, glycolipids, bacterial cell walls, subunits (bacterial or viral), carbohydrate moieties (mono-, di, tri-, tetra-, oligo- and polysaccharide), various liposome formulations or saponins. Combinations of various adjuvants may be used with the antigen to prepare the immunogen formulations. Adjuvants administered parentally or for the induction of mucosal immunity may also be used.

The MSP2 peptides of the invention comprise the semi-conserved family-specific domain plus the C-terminal domain that is highly conserved in all MSP2 alleles. As shown in the Examples, antigenicity studies on immune sera from different malaria endemic areas and different age groups showed a high prevalence of antibody recognizing both long synthetic peptides. Furthermore, prevalences of antibodies to recombinant proteins corresponding to the family-specific parts and the conserved parts were in accordance with results obtained with peptides. All of these findings indicate similar structural and immunological properties of recombinant antigens and synthetic MSP2 peptides. In addition, affinity-purified antibodies react with native MSP2 on the surface of merozoites as shown in FIG. 3. Taken together these data provide evidence for the peptides of the invention (synthetic or recombinant) to share major epitopes with parasite-derived MSP2. This may be due to the fact that MSP2 is an intrinsically unstructured protein as indicated by the low amino acid complexity of its sequence.

The present invention therefore also contemplates a vaccine composition useful to stimulate an immune response in a mammal characterized in that it comprises an antigenic peptide of the invention, or an antigenic cocktail composition of the invention or an antibody of the invention. Preferably, the vaccine composition further comprises an adjuvant; said adjuvant can be selected from the list described above.

The vaccine composition can be administered by various delivery methods including intravascularly, intraperitoneally, intramuscularly, intradermally, subcutaneously, orally, nasally or by inhalation. In an embodiment, the compositions can further include a pharmaceutically acceptable excipient and/or carrier.

The vaccine composition may comprise one or several doses of the peptide or antigenic cocktail or antibody of the invention, the quantity of one dose being determined and/or adjusted by the physician taking into account the patient's health, notably of the state of the patient's immunity system, and taking into account the patient's medical features, such as age and weight. Illustrative doses are between 8 to 100 μg per adult person, preferably between 9 to 60 μg per adult person, e.g. 10, 25 or 50 μg per adult person.

When formulated as a multidose composition, the vaccine composition of the invention may for example comprise said at least one antigenic peptide, or antigenic cocktail, or antibody of the invention in a quantity corresponding to 2 to 20 individual doses, said individual doses being either mixed together in the same container or vial or contained in individual containers or vials.

The administration schedule is to be determined by the physician depending on the patient's health, the stage of the Plasmodium infection, the patient's age, the patient's weight, and of the dose to be administered or that has already been administered. Typically, two or three doses at monthly interval are expected to be efficient in the treatment, more specifically the palliative and/or curative treatment of the disease.

“Administered” or “administering”, as it applies in the present invention, refers to contact of a pharmaceutical, therapeutic, diagnostic agent or composition, to the subject, preferably a human.

The exact formulation of the vaccine composition will depend on the particular antigenic peptide, or an antigenic cocktail, or peptide-carrier conjugate, or antibody and the route of administration.

When employing more than one antigenic peptide, such two or more, or an antigenic cocktail composition, they may be used as a physical mixture or a fusion of two or more antigenic peptides to form a combination. The combination may be produced, for example, by recombinant techniques, by the use of appropriate linkers for fusing previously prepared antigenic peptides or by co-linearly synthesizing the combination with or without linkers such as, for example, PEG (poly ethylene glycol).

Also encompassed in the present invention is an antibody characterized in that it recognizes the antigenic peptide of the invention or the antigenic cocktail composition of the invention.

As used herein, an “antibody” is a protein molecule that reacts with a specific antigen and belongs to one or five distinct classes based on structural properties: IgA, IgD, IgE, IgG and IgM. The antibody may be a polyclonal (e.g. a polyclonal serum) or a monoclonal antibody, including but not limited to fully assembled antibody, single chain antibody, antibody fragment, and chimeric antibody, humanized antibody as long as these molecules are still biologically active and still bind to at least one peptide of the invention.

Examples of the fragment of an antibody include Fab, F(ab′)2, Fv, Fab/c having one Fab and a complete Fc, and a single chain Fv (scFv) wherein the Fv of the H-chain or the L-chain is liagted with an appropriate linker. Specifically, an antibody fragment is synthesized by treating the antibody with an enzyme such as papain or pepsin, or genes encoding these antibody fragments are constructed, and expressed by appropriate host cells as known to the skilled artisan. Preferably the antibody is a monoclonal antibody. Preferably also the antibody will be selected from the group comprising the IgG1, IgG2, IgG2a, IgG2b, IgG3 and IgG4.

As shown in the examples, the immunogenicity of peptides MR141 and MR144A were tested in mice. Both the family-specific and C-terminal conserved regions present on the peptides MR141 and MR144A elicited high titre IgG responses in CB6F1 mice when administered with Montanide ISA 720. A positive response to this region was observed in children but not in adults, suggesting anergic properties of this part of the molecule with increasing exposure (Thomas et al.; Stowers et al.; Lawrence et al.). Also, vaccination with Combination B did not induce a response to the conserved regions of MSP2 (Fluck et al.). Monoclonal antibodies directed to both the 3D7 family-specific and the C-terminal conserved part in peptide MR141 were generated. These monoclonal antibodies were also reactive to the merozoite surface, confirming that a cross-reactivity to native MSP2 was elicited.

Applicants evaluated protection in relation to total IgG level and both IgG1 and IgG3 against the peptides MR141 (3D7) and MR144A (FC27).

Despite the limited sample size of 280 children, Applicant's analysis also suggests that antibodies to the 3D7 MSP2 fragment are associated with protection to malaria infection. The relationship is strongest with levels of IgG3. No association with protection was observed for the FC27 MSP2 fragment. This could be related to the fact that the response to FC27 MSP2 is skewed towards the highly polymorphic repeat region.

The antibodies of the invention can also be produced for use in passive immunotherapy, for use as diagnostic reagents, and for use as reagents in other processes such as affinity chromatography.

When used in passive immunotherapy, the antibody of the invention can be included in a pharmaceutical composition or a vaccine composition and administered to a mammal. In an embodiment, the pharmaceutical composition includes a pharmaceutically acceptable carrier, and optionally can include pharmaceutically acceptable excipients. The pharmaceutical composition can be administered intravascularly, intraperitoneally, intramuscularly, intradermally, subcutaneously, orally, nasally or by aerosol inhalation. Preferably, the pharmaceutical composition is administered intravascularly, intramuscularly, orally, nasally or by aerosol inhalation.

The quantity of one dose being determined and/or adjusted by the physician taking into account the patient's health, notably of the state of the patient's immunity system, and taking into account the patient's medical features, such as age and weight.

The administration schedule is to be determined by the physician depending on the patient's health, the stage of the Plasmodium infection, the patient's age, the patient's weight, and of the dose to be administered or that has already been administered. Typically, two or three doses at monthly interval are expected to be efficient in the treatment.

In an embodiment, the present invention includes an antibody, particularly a monoclonal antibodies, directed to the antigenic peptide or the antigenic cocktail composition of the invention. In particular, hybridomas can be generated using a peptide of the invention and recombinant derivative antibodies can be made using these hybridomas according to well-known genetic engineering methods (Winter G. and Milstein C.). Preferably, as disclosed in example 2 and Table 2 or Table 3, the antibody is selected from the group comprising the IgG1, IgG2, IgG3 and IgG4 or mixtures thereof.

Other methods known in the art to humanize an antibody or produce a humanized antibody can be utilized as well. These methods can include but are not limited to the xenomouse technology developed by ABGENIX INC. (See, U.S. Pat. Nos. 6,075,181 and 6,150,584) and the methods developed by BIOVATION, BIOINVENT INTERNATIONAL AB, PROTEIN DESIGN LABS, APPLIED MOLECULAR EVOLUTION, INC., IMMGENICS PHARMACEUTICALS INC., MEDAREX INC., CAMBRIDGE ANTIBODY TECHNOLOGY, ELAN, EOS BIOTECHNOLOGY, MEDIMMUNE, MORPHOSYS, UROGENSYS INC., AVANIR PHARMACEUTICAL/XENEREX BIOSCIENCES, AFFIBODY AB, ALLEXION ANTIBODY TECHNOLOGIES, ARIUS RESEARCH INC., CELL TECH, XOMA, IDEC PHARMACEUTICALS, NEUGENESIS, EPICYTE, SEMBIOSYS GENETICS INC., BIOPROTEIN, GENZYME THERAPEUTICS, KIRIN, GEMINI SCIENCES, HEMATECH.

Likewise, other methods known in the art to screen human antibody secreting cells to the peptide of the invention may be also utilized.

Also encompassed by the present invention is a hybridoma expressing an antibody according to the invention. As used herein “hybridoma” are cells that have been engineered to produce a desired antibody in large amounts. To produce monoclonal antibodies, B-cells are removed from the spleen of an animal that has been challenged with the relevant antigen. These B-cells are then fused with myeloma tumor cells that can grow indefinitely in culture (myeloma is a B-cell cancer). This fusion is performed by making the cell membranes more permeable. The fused hybrid cells (called hybridomas), being cancer cells, will multiply rapidly and indefinitely and will produce large amounts of the desired antibodies. They have to be selected and subsequently cloned by limiting dilution; this procedure is well known from the skilled artisan.

As used herein, the term “humanized antibody” or other like terms means an antibody that includes a human protein sequence in at least a portion thereof. The amount of human protein sequence can vary depending on how the antibody is made.

A “fully humanized antibody” or “human antibody” as the terms or like terms are used herein can be made, for example, with xenomouse technology as discussed above or transforming human B cells with Epstein Barr virus (Traggiai et al.). Other methods such as phage display techniques are also possible (Bradbury A R and Marks J D,).

The term “recognize” refers to the fact that an antibody of the invention is directed to an antigenic peptide or an antigenic cocktail composition of the invention and binds thereto.

The antibody of the present invention may also be used in combination with other therapeutic agents such as proteins, antibodies, and/or with targeting molecules to specifically target a certain cell type, and/or to detection label, such as radio-isotope to easily detect said antibody.

When recombinant techniques are employed to prepare an antigenic peptide in accordance with the present invention, nucleic acid molecules or fragments thereof encoding the polypeptides are preferably used.

Therefore the present invention also relates to a purified and isolated nucleic acid sequence comprising
i) a nucleotide sequence encoding an antigenic peptide of the invention,
ii) a nucleic acid sequence complementary to i),
iii) a degenerated nucleic acid sequence of i) or ii),
iv) a nucleic acid sequence capable of hybridizing under stringent conditions to i), ii) or iii),
v) a nucleic acid sequence encoding a truncation or an analog of an antigenic peptide of the invention,
vi) and/or a fragment of i), ii), iii), iv) or v) encoding a biologically active fragment of said antigenic peptide of the invention.

“A purified and isolated nucleic acid sequence” refers to the state in which the nucleic acid molecule encoding the antigenic peptide of the invention, or nucleic acid encoding such the antigenic peptide will be, in accordance with the present invention. Nucleic acid will be free or substantially free of material with which it is naturally associated such as other polypeptides or nucleic acids with which it is found in its natural environment, or the environment in which it is prepared (e.g. cell culture) when such preparation is by recombinant nucleic acid technology practised in vitro or in vivo.

The term “nucleic acid” is intended to refer either to DNA or to RNA.

In case the nucleic acid is DNA, then DNA which can be used herein is any polydeoxynucleotide sequence, including, e.g. double-stranded DNA, single-stranded DNA, double-stranded DNA wherein one or both strands are composed of two or more fragments, double-stranded DNA wherein one or both strands have an uninterrupted phosphodiester backbone, DNA containing one or more single-stranded portion(s) and one or more double-stranded portion(s), double-stranded DNA wherein the DNA strands are fully complementary, double-stranded DNA wherein the DNA strands are only partially complementary, circular DNA, covalently-closed DNA, linear DNA, covalently cross-linked DNA, cDNA, chemically-synthesized DNA, semi-synthetic DNA, biosynthetic DNA, naturally-isolated DNA, enzyme-digested DNA, sheared DNA, labeled DNA, such as radiolabeled DNA and fluorochrome-labeled DNA, DNA containing one or more non-naturally occurring species of nucleic acid.

DNA sequences that encode the antigenic peptide of the invention, or a fragment thereof, can be synthesized by standard chemical techniques, for example, the phosphotriester method or via automated synthesis methods and PCR methods.

The purified and isolated DNA sequence encoding the antigenic peptide according to the invention may also be produced by enzymatic techniques. Thus, restriction enzymes, which cleave nucleic acid molecules at predefined recognition sequences can be used to isolate nucleic acid sequences from larger nucleic acid molecules containing the nucleic acid sequence, such as DNA (or RNA) that codes for the antigenic peptide of the invention or for a fragment thereof.

Encompassed by the present invention is also a nucleic acid in the form of a polyribonucleotide (RNA), including, e.g., single-stranded RNA, double-stranded RNA, double-stranded RNA wherein one or both strands are composed of two or more fragments, double-stranded RNA wherein one or both strands have an uninterrupted phosphodiester backbone, RNA containing one or more single-stranded portion(s) and one or more double-stranded portion(s), double-stranded RNA wherein the RNA strands are fully complementary, double-stranded RNA wherein the RNA strands are only partially complementary, covalently crosslinked RNA, enzyme-digested RNA, sheared RNA, mRNA, chemically-synthesized RNA, semi-synthetic RNA, biosynthetic RNA, naturally-isolated RNA, labeled RNA, such as radiolabeled RNA and fluorochrome-labeled RNA, RNA containing one or more non-naturally-occurring species of nucleic acid.

The purified and isolated nucleic acid sequence, DNA or RNA, also comprises a purified and isolated nucleic acid sequence having substantial sequence identity or homology to a nucleic acid sequence encoding an antigenic peptide of the invention. Preferably, the nucleic acid will have substantial sequence identity for example at least 50%, 55%, 60%, 65%, 70%, 75%, 80%, or 85% nucleic acid identity; more preferably 90% nucleic acid identity; and most preferably at least 95%, 96%, 97%, 98%, or 99% sequence identity.

“Identity” as known in the art and used herein, is a relationship between two or more amino acid sequences or two or more nucleic acid sequences, as determined by comparing the sequences. It also refers to the degree of sequence relatedness between amino acid or nucleic acid sequences, as the case may be, as determined by the match between strings of such sequences. Identity and similarity are well known terms to skilled artisans and they can be calculated by conventional methods (for example see Computational Molecular Biology, Lesk, A. M. ed; Biocomputing: Informatics and Genome Projects, Smith, D. W. ed.,; Computer Analysis of Sequence Data, Part I, Griffin, A. M. and Griffin, H. G.; Sequence Analysis in Molecular Biology, von Heinje, G.; and Sequence Analysis Primer, Gribskov, M. and Devereux; Carillo, H. and Lipman, D.).

Methods which are designed to give the largest match between the sequences are generally preferred. Methods to determine identity and similarity are codified in publicly available computer programs including the GCG program package (Devereux J. et al.,); BLASTP, BLASTN, and FASTA (Altschul, S. F. et al.). The BLAST X program is publicly available from NCBI and other sources (BLAST Manual, Altschul, S. et al. NCBI NLM NIH Bethesda, Md. 20894).

Also encompassed by the present invention is a nucleic acid sequence complementary to the antigenic peptide of the invention.

Also within the scope of the invention is a degenerated nucleic acid sequence having a sequence which differs from a nucleic acid sequence encoding the antigenic peptide of the invention, or a complementary sequence thereof, due to degeneracy in the genetic code. Such nucleic acid encodes functionally equivalent to the antigenic peptide of the invention but differs in sequence from the sequence due to degeneracy in the genetic code. This may result in silent mutations which do not affect the amino acid sequence. Any and all such nucleic acid variations are within the scope of the invention.

In addition, also considered is a nucleic acid sequence capable of hybridizing under stringent conditions, preferably high stringency conditions, to a nucleic acid sequence encoding the antigenic peptide of the invention, a nucleic acid sequence complementary thereof or a degenerated nucleic acid sequence thereof. Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0× sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

The present invention also includes a purified and isolated nucleic acid encoding an antigenic peptide of the invention comprising a nucleic acid sequence encoding a truncation or an analog of the antigenic peptide.

The invention also encompasses allelic variants of the disclosed purified and isolated nucleic sequence; that is, naturally-occurring alternative forms of the isolated and purified nucleic acid that also encode antigenic peptides that are identical, homologous or related to that encoded by the purified and isolated nucleic sequences. Alternatively, non-naturally occurring variants may be produced by mutagenesis techniques or by direct synthesis.

A fragment of the disclosed purified and isolated nucleic sequence is also considered and refers to a sequence containing less nucleotides in length than the nucleic acid sequence encoding the antigenic peptide, a nucleic acid sequence complementary thereof or a degenerated nucleic acid sequence thereof. This sequence can be used as long as it exhibits the same properties as the native sequence from which it derives. Preferably this sequence contains less than 90%, preferably less than 60%, in particular less than 30% amino acids in length than the respective purified and isolated nucleic sequence of the antigenic peptide.

Yet another concern of the present invention is to provide an expression vector comprising at least one copy of the purified and isolated nucleic acid sequence encoding an antigenic peptide of the invention as described above.

The choice of an expression vector depends directly, as it is well known in the art, on the functional properties desired, e.g., antigenic peptide expression and the host cell to be transformed or transfected.

Additionally, the expression vector may further comprise a promoter operably linked to the purified and isolated nucleic acid sequence of the invention. This means that the linked isolated and purified nucleic acid sequence encoding the antigenic peptide of the present invention is under control of a suitable regulatory sequence which allows expression, i.e. transcription and translation of the inserted isolated and purified nucleic acid sequence.

As used herein, the term “promoter” designates any additional regulatory sequences as known in the art e.g. a promoter and/or an enhancer, polyadenylation sites and splice junctions usually employed for the expression of the polypeptide or may include additionally one or more separate targeting sequences and may optionally encode a selectable marker. Promoters which can be used provided that such promoters are compatible with the host cell are e.g promoters obtained from the genomes of viruses such as polyoma virus, adenovirus (such as Adenovirus 2), papilloma virus (such as bovine papilloma virus), avian sarcoma virus, cytomegalovirus (such as murine or human cytomegalovirus immediate early promoter), a retrovirus, hepatitis-B virus, and Simian Virus 40 (such as SV 40 early and late promoters) or promoters obtained from heterologous mammalian promoters, such as the actin promoter or an immunoglobulin promoter or heat shock promoters.

Enhancers which can be used are e.g. enhancer sequences known from mammalian genes (globin, elastase, albumin, a-fetoprotein, and insulin) or enhancer from a eukaryotic cell virus e.g. the SV40 enhancer, the cytomegalovirus early promoter enhancer, the polyoma, and adenovirus enhancers.

A wide variety of host/expression vector combinations may be employed in expressing the nucleic acid sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMB9 and their derivatives, plasmids such as RP4; phage DNAs, e.g., the numerous derivatives of phage X, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Another concern of the present invention is to provide a host cell (eukaryotic or prokaryotic) comprising a purified and isolated nucleic acid sequence of the invention or an expression vector as described above.

Typically, this host cell has been transformed or transfected with a purified and isolated nucleic acid sequence of the invention or an expression vector described herein.

The term “cell transfected” or “cell transformed” or “transfected/transformed cell” means the cell into which the extracellular DNA has been introduced and thus harbours the extracellular DNA. The DNA might be introduced into the cell so that the nucleic acid is replicable either as a chromosomal integrant or as an extra chromosomal element.

Transformation or transfection of appropriate eukaryotic or prokaryotic host cells with an expression vector comprising a purified an isolated DNA sequence according to the invention is accomplished by well known methods that typically depend on the type of vector used. With regard to these methods, see e.g., Maniatis et al. 1982, Molecular Cloning, A laboratory Manual, Cold Spring Harbor Laboratory and commercially available methods.

A wide variety of unicellular host cells are useful in expressing the nucleic acid sequences of this invention. These hosts may include well known eukaryotic and prokaryotic hosts, such as strains of E. coli, Pseudomonas, Bacillus, Streptomyces, fungi such as yeasts, and animal cells, such as CHO, YB/20, NSO, SP2/0, R1. 1, B-W and L-M cells, African Green Monkey kidney cells (e.g., COS 1, COS 7, BSC1, BSC40, and BMT10), insect cells (e.g., Sf9), and human cells and plant cells in tissue culture. Preferably, the host cell is a bacterial cell, more preferably an E. coli cell.

The present invention is also directed to a vaccine composition for the treatment and/or prevention of malaria comprising at least one antigenic peptide of the invention, or the antigenic cocktail composition of the invention.

The vaccine of the invention may also contain several antibodies or antibody fragments of the invention, e.g. two or three antibodies or antibody fragments of the invention

In a further aspect, the present invention is also directed to an acid nucleic vaccine composition for the treatment and/or prevention of malaria comprising at least one purified and isolated nucleic acid sequence of the invention, or an expression vector comprising at least one copy of the purified and isolated nucleic acid sequence of the invention, fragments thereof, molecular chimeras thereof, combinations thereof and/or variants thereof.

The present invention also encompasses a diagnostic tool for determining the presence of an antigenic peptide or an antigenic cocktail composition of the invention in a sample comprising:
i) contacting the sample with an antibody directed to the antigenic peptide or the antigenic cocktail composition of the invention, and
ii) determining whether said antibody binds to a component of said sample.

The present invention further encompasses a diagnostic tool for determining the presence of antibodies directed to the antigenic peptide or to the antigenic cocktail composition of the invention in a sample comprising:
i) contacting said sample with the antigenic peptide or the antigenic cocktail composition of the invention, and
ii) determining whether antibodies bind to a component of said antigenic peptide or to said antigenic cocktail composition of the invention.

As used herein, “a sample” is an aliquot or a representative portion of a substance, material or population. For example, a sample may be a sample of blood, biological tissue, urine or feces. Preferably, the sample is blood.

As used herein, “a donor” is an individual person that lives in pathogen endemic areas and that has been, or is suspected to be, infected by said pathogen.

A “component” refers to any amino acid, nucleic acid, lipid or motif that is recognized by the antibody of the invention.

Further encompassed is a protein characterized in that it comprises at least one antigenic peptide of the invention or an antigenic cocktail composition of the invention.

Also within the scope of the present invention is a kit, said kit comprising the vaccine composition as described herein, optionally with reagents and/or instructions for use. Alternatively, or additionally, the kit may further include other materials desirable from a commercial and user standpoint, including buffers, diluents, filters, needles, and syringes.

Various references are cited throughout this Specification, each of which is incorporated herein by reference in its entirety.

The foregoing description will be more fully understood with reference to the following Examples. Such Examples, are, however, exemplary of methods of practising the present invention and are not intended to limit the scope of the invention.

### REFERENCE LIST

Alonso, P. L., et al. “Randomised trial of efficacy of SPf66 vaccine against Plasmodium falciparum malaria in children in southern Tanzania.” Lancet 344 (1994).
Alpers, M. P., et al. “The Malaria Vaccine Epidemiology and Evaluation Project of Papua New Guinea: rationale and baseline studies.” P N G Med J 35. (4) (1992).
Altschul, S. F. et al. J. Molec. Biol. 215: 403-410, (1990).
Beck, H. P., et al. “Humoral and cell-mediated immunity to the Plasmodium falciparum ring-infected erythrocyte surface antigen in an adult population exposed to highly endemic malaria.” Infect Immun 63(2) (1995).
Bouharoun-Tayoun H, et al. Antibodies that protect humans against Plasmodium falciparum blood stages do not on their own inhibit parasite growth and invasion in vitro, but act in cooperation with monocytes. J Exp Med 1990; 172(6):1633-41
Bradbury A R, Marks J D, J. immunol. Methods, 290 (1-2): 29-49 (2004).
Carillo, H. and Lipman, D., SIAM J. Applied Math. 48:1073, (1988).
Cowman, A. F., et al. “Functional analysis of proteins involved in Plasmodium falciparum merozoite invasion of red blood cells.” FEBS Lett 476.1-2 (2000).
Devereux J. et al., Nucleic Acids Research 12(1): 387, (1984);
Epping, R. J., et al. “An epitope recognised by inhibitory monoclonal antibodies that react with a 51 kilodalton merozoite surface antigen in Plasmodium falciparum.” Mol Biochem Parasitol 28(1) (1988).
Fluck, C., et al. “Strain-specific humoral response to a polymorphic malaria vaccine.” Infect Immun 72(11) (2004).
Fraser-Hurt, N., et al. “Effect of insecticide-treated bed nets on haemoglobin values, prevalence and multiplicity of infection with Plasmodium falciparum in a randomized controlled trial in Tanzania.” Trans R Soc Trop Med Hyg 93 Suppl 1 (1999).
Genton, B., et al. “Safety and immunogenicity of a three-component blood-stage malaria vaccine (MSP1, MSP2, RESA) against Plasmodium falciparum in Papua New Guinean children.” Vaccine 22(1) (2003).
Genton, B., et al. “A recombinant blood-stage malaria vaccine reduces Plasmodium falciparum density and exerts selective pressure on parasite populations in a phase 1-2b trial in Papua New Guinea.” J Infect Dis 185(6) (2002).
Gribskov, M. and Devereux, J. Sequence Analysis Primer, eds. M. Stockton Press, New York, (1991)
Griffin, A. M. and Griffin, H. G Computer Analysis of Sequence Data, Part I, eds., Humana Press, New Jersey, (1994)
von Heinje, G Sequence Analysis in Molecular Biology, Academic Press, (1987)
Irion, A. “Molecular diversity and immunological properties of the Plasmodium falciparum merozoite surface protein 2 (MSP2).” PhD Thesis (2000).
Irion, A., H. P. Beck, and T. Smith. “Assessment of positivity in immuno-assays with variability in background measurements: a new approach applied to the antibody response to Plasmodium falciparum MSP2.” J Immunol Methods 259.1-2 (2002).
Jones, G. L., et al. “Immunological fine structure of the variable and constant regions of a polymorphic malarial surface antigen from Plasmodium falciparum.” Mol Biochem Parasitol 48(1) (1991).
Lambros, C. and J. P. Vanderberg. “Synchronization of Plasmodium falciparum erythrocytic stages in culture.” J Parasitol 65(3) (1979).
Lawrence, G., et al. “Effect of vaccination with 3 recombinant asexual-stage malaria antigens on initial growth rates of Plasmodium falciparum in non-immune volunteers.” Vaccine 18(18) (2000).
Lawrence, N., et al. “Recombinant chimeric proteins generated from conserved regions of Plasmodium falciparum merozoite surface protein 2 generate antiparasite humoral responses in mice.” Parasite Immunol 22.5 (2000).
Lesk, A. M Computational Molecular Biology, Oxford University Press, New York, (1988)
Maniatis et al., Molecular Cloning, A laboratory Manual, Cold Spring Harbor Laboratory (1982)
McGregor, I. et al. “Treatment of East African Plasmodium falciparum malaria with West African gammaglobulin.” Trans R Soc Trop Med Hyg (1963).
Metzger, W. G., et al. “Serum IgG3 to the Plasmodium falciparum merozoite surface protein 2 is strongly associated with a reduced prospective risk of malaria.” Parasite Immunol 25(6) (2003).
Sabchareon, A., et al. “Parasitologic and clinical human response to immunoglobulin administration in falciparum malaria.” Am J Trop Med Hyg 45(3) (1991).
Saul, A., et al. “Protective immunization with invariant peptides of the Plasmodium falciparum antigen MSA2.” J Immunol 148.1 (1992). Erratum in: J Immunol, 154:4223 (1995)
Sela M. and Zisman E., Different roles of D-amino acids in immune phenomena—FASEB J. 11, 449 (1997)
Smith, T., et al. “Absence of seasonal variation in malaria parasitaemia in an area of intense seasonal transmission.” Acta Trop 54(1) (1993).
Smith, D. W Biocomputing: Informatics and Genome Projects, ed., Academic Press, New York, (1993)
Stowers, A., et al. “Assessment of the humoral immune response against Plasmodium falciparum rhoptry-associated proteins 1 and 2.” Infect Immun 65(6) (1997).
Taylor, R. R., et al. “IgG3 antibodies to Plasmodium falciparum merozoite surface protein 2 (MSP2): increasing prevalence with age and association with clinical immunity to malaria.” Am J Trop Med Hyg 58(4) (1998).
Taylor, R. R., et al. “Human antibody response to Plasmodium falciparum merozoite surface protein 2 is serogroup specific and predominantly of the immunoglobulin G3 subclass.” Infect Immun 63(11) (1995).
Thomas, A. W., et al. “Sequence comparison of allelic forms of the Plasmodium falciparum merozoite surface antigen MSA2.” Mol Biochem Parasitol 43(2) (1990).
Traggiai et al, Nat. Med. 10(8): 871-5 (2004).
Trager, W. and J. B. Jensen. “Human malaria parasites in continuous culture.” Science 193 (4254) (1976).
Winter G., and Milstein C., Nature, 349, 293-299 (1991)

The BLAST X program is publicly available from NCBI and other sources (BLAST Manual, Altschul, S. et al. NCBI NLM NIH Bethesda, Md. 20894).

### EXAMPLES

### Example 1

### Material and Methods

### Human Sera

Sample set I: Sera were collected in the Maprik District of the East Sepik Province, Papua New Guinea, during a cross sectional survey in July 1992 within the framework of the Malaria Vaccine Epidemiology and Evaluation Project (MVEEP) supported by the United States Agency for International Development (Alpers et al.) (Beck et al.). The area is highly endemic for malaria. Ethical clearance for MVEEP was obtained from the PNG Medical Research Advisory Committee. Blood was taken by venipuncture into tubes containing EDTA. Sera of twenty of these subjects were pooled and used as positive control in enzyme linked immunosorbent assays (ELISAs). For affinity purification of human anti-MSP2 antibodies, sera of 18 adults of both sexes were pooled.
Sample set II: The sera were collected in the village of Goundry located in the central Mossi Plateau, between 15 and 50 km north of the capital Ouagadougou, in the province of Oubritenga. The climate is characteristic of areas of Sudanese savannah, with a dry season from November to May and a rainy season from June to October. Malaria transmission is very high during the rainy season and markedly seasonal. Ethical clearance was obtained from the Ministry of Health, Burkina Faso. After obtaining informed consent from parents and caretakers, heparinized venous blood samples were collected during a cross-sectional survey during the malaria low transmission season 1998.
Sample set III: Sera had been collected in the course of a study on effects of insecticide-treated bednets on malariological parameters, antibody responses and multiplicity of P. falciparum infections in infants in the village of Kiberege, Kilombero District, southern Tanzania (Fraser-Hurt et al.). The area is holoendemic for malaria with approximately 300 infectious bites per year with perennial transmission and little seasonal changes of parasite prevalence and density (Smith et al.). Two finger-prick blood samples were collected seven months apart from 60 children initially aged 5 to 15 months.
Sample set IV: Sera originate from a SPf-66 efficacy trial conducted in Tanzanian children 1 to 5 years of age (Alonso et al.). Two hundred and eighty baseline sera of placebo recipients were used for ELISA and morbidity data from the one year follow up period during the vaccine trial was used to analyse protection against clinical malaria with respect to antibody titres against synthetic MSP2 peptides.

Research and ethical clearance for the Tanzanian studies were granted by the Tanzanian Commission for Science and Technology (NSR/RCA 90).

### Synthetic Peptides

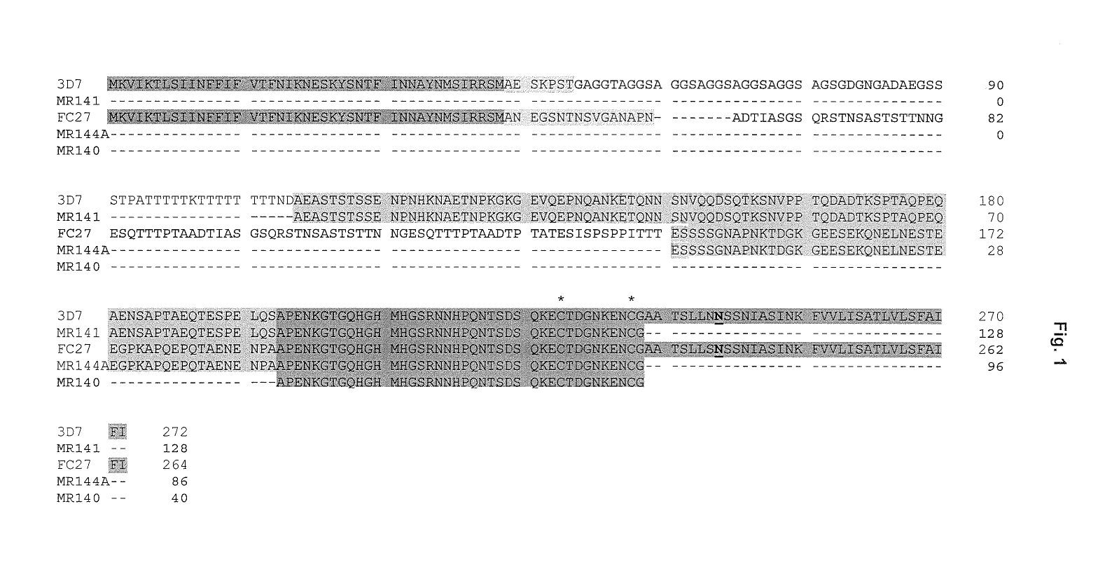

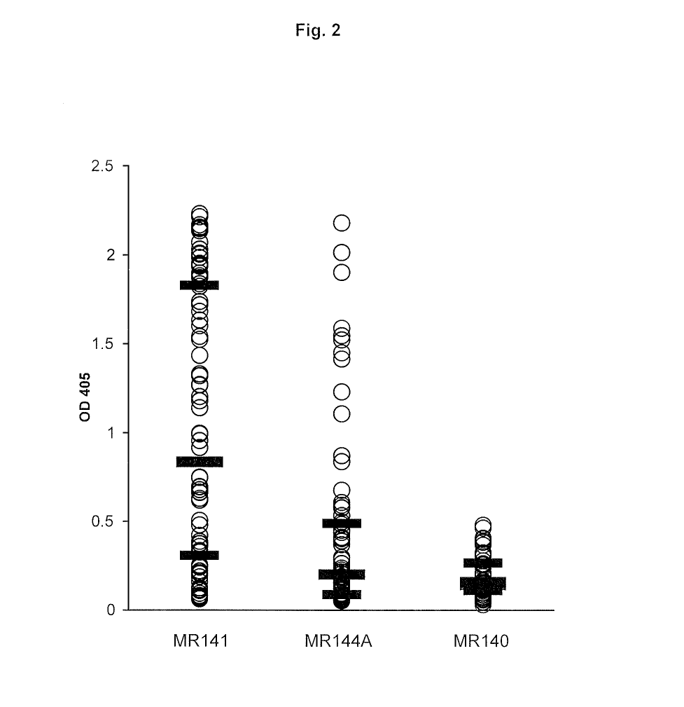

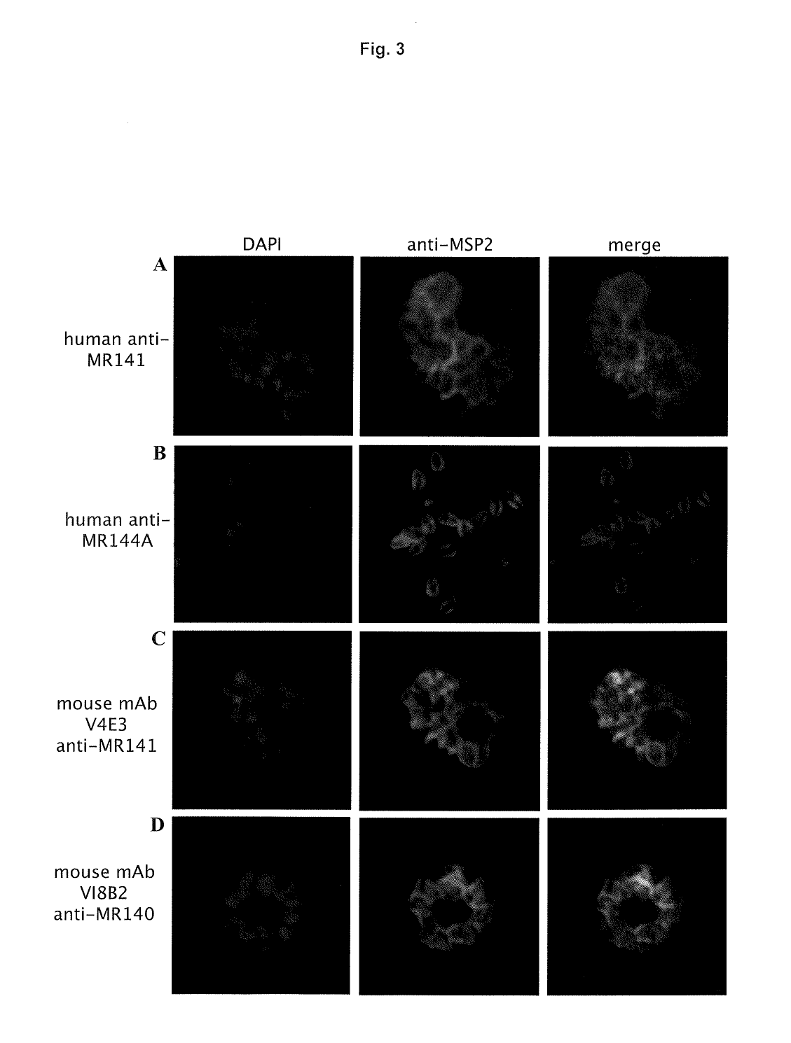

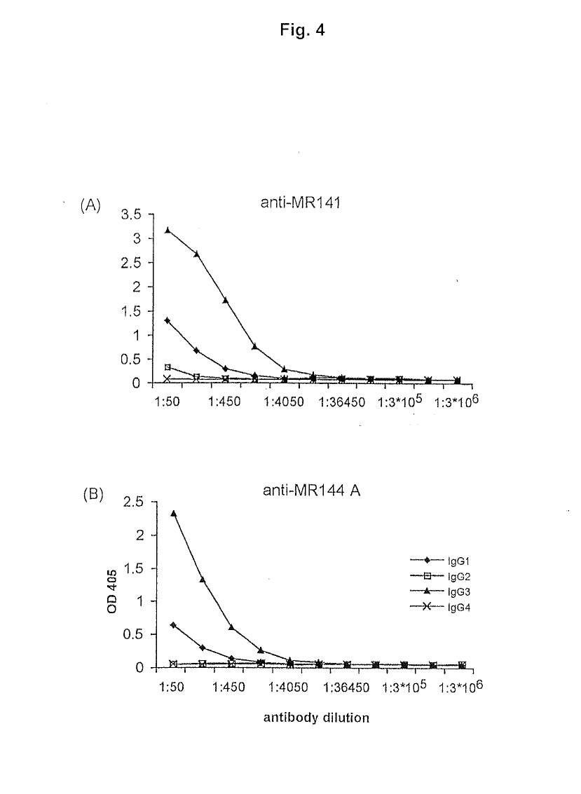

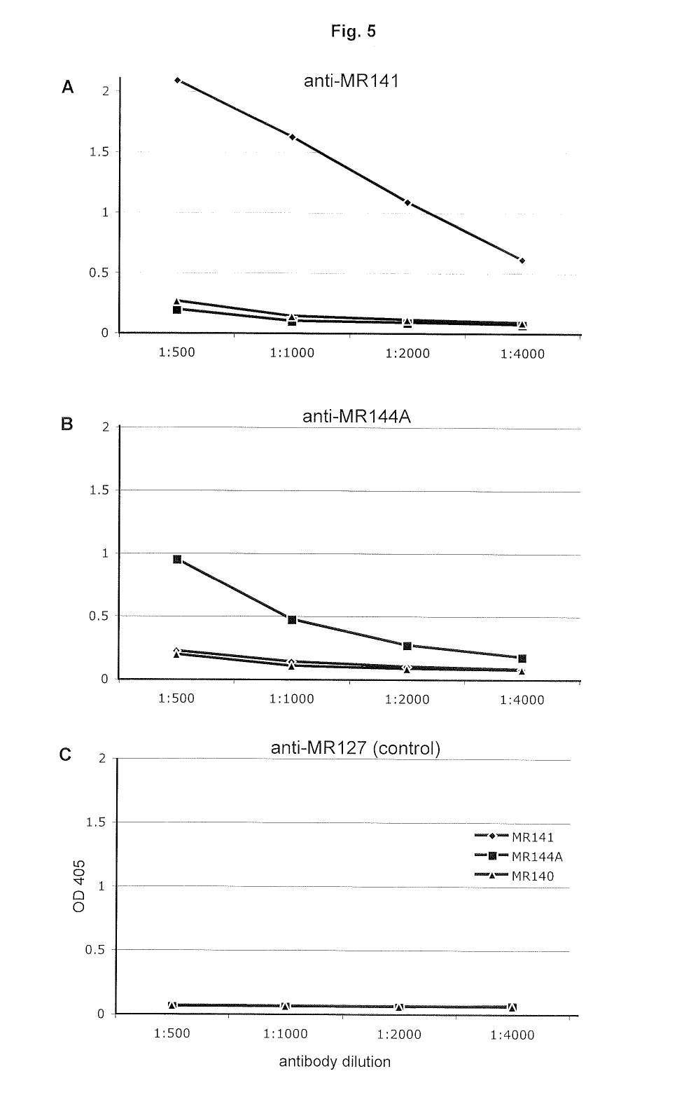

Peptides (FIG. 1) were synthesized in an Applied Biosystem 431A (Foster City, Calif.) using solid-phase Fmoc chemistry. Briefly, peptides were prepared on a p-alkoxybenzylalcohol resin (Wang resin). After cleavage from the resin, the crude peptide was purified by RP-HPLC (C18 preparative column using a gradient of 0.1% TFA in H₂O and 0.1% TFA in acetonitrile. The purity (>80%) was determined by analytical C18 HPLC and mass spectroscopy (MALDI-TOF, Applied Biosystem). Lyophilised peptides were dissolved in phosphate buffered saline (PBS) at a concentration of 1 mg/ml.

Immunization of Mice with Long Synthetic MSP2 peptides MR141 (SEQ ID No 1) (3D7 MSP2) and MR144A (SEQ ID No 3) (FC27 MSP2)

Groups of five mice each were immunized three times subcutaneously with 20 μg of peptide

MR141 (SEQ ID No 1) (3D7 MSP2) or MR144A (SEQ ID No 3) (FC27 MSP2), respectively in 50 μl adjuvant. Group 1: BALB/c mice, 20 μg peptide MR141+Montanide ISA 720; Group 2: CB6F1 mice, 20 μg peptide MR141+Montanide ISA 720; Group 3: BALB/c mice, 20 μg peptide MR141+ incomplete Freund's adjuvant (IFA); Group 4: CB6F1 mice, 20 μg peptide MR144A+Montanide ISA 720.

Production of Monoclonal Antibodies (mAbs)

Mice giving the highest titres against the immunogen were chosen for production of monoclonal antibodies (mAbs). Mice were injected a fourth time intraperitoneally with 1 μg of the peptide in 100 μl PBS. Three days after the fourth immunization, spleens were sterilely removed and fused with the mouse myeloma cell line X63.Ag8.653. Culture supernatants of growing hybrids were screened for antibodies by ELISA and indirect immunofluorescence assays (IFA). Positive hybrids, based on ELISA and IFA were cloned by limiting dilution.

Hybridoma clones secreting the monoclonal antibodies of interest were grown in serum-free OPTIMEM medium.

### Enzyme Linked Immunosorbant Assay (ELISA)

Recognition of the synthetic peptides by human sera and monoclonal antibodies was assessed by ELISA. The optimal coating concentration of the peptides was determined by a checkerboard titration with positive and negative control sera. The optimal concentration was 1 μg/ml for MR141 and MR144A, and 5 μg/ml for MR140.

Immulon® 2HB plates (Thermo Labsystems, Beverly, Mass.) were coated overnight with 500 of peptide at the appropriate concentration. Plates were blocked for one hour at room temperature in phosphate buffered saline (PBS) containing 5% non fat milk powder. Antibody reactions were carried out in PBS containing 0.5% milk powder and 0.05% Tween 20. Human sera were diluted 1:400 for assays on MR140 and 1:1000 for assays on MR141, and MR144A (sample sets I and III) or serially diluted 1:3 (sample set II). Supernatants from hybridoma cultures were also serially diluted 1:3. A serum pool of 20 semi-immune adults from PNG was used as internal standard. Pooled sera from 40 non-exposed European children aged 5-15 months were used to determine the cut off value of our ELISAs. The plates were incubated for two hours at room temperature. Plate washing was performed in an ELISA washer with water containing 0.05% Tween 20. Secondary antibodies were incubated for two hours at room temperature. Goat anti-human IgG-γ specific HRP-conjugated antibody from Kirkegaard and Perry Laboratories (KPL, Gaithersburg, Mass.) was used at a dilution of 1:2000 and Goat anti-mouse IgG (heavy+light chain) HRP-conjugated antibody from Bio-rad (Hercules, Calif.) was used at a 1:5000 dilution. After extensive washing, ABTS peroxidase substrate (KPL) was added. The reaction was stopped after 30 minutes with 1% sodium dodecyl sulfate and the plates were read at 405 nm. For IgG subclass-specific ELISAs alkaline phosphatase (AP)-labelled anti-human IgG1, IgG3, and IgG4 antibodies (Southern Biotech, Birmingham, Ala.) were used at a dilution of 1:1000 and an AP-labelled anti-human IgG2 antibody (Zymed, Invitrogen, Carlsbad, Calif.) was used at a dilution of 1:500.
Affinity Purification of Antibodies from Human Sera and Hybridoma Supernatants

5 mg of each peptide (MR141, MR144A) were coupled to CNBr-activated sepharose with a final column volume of 1 ml. The column was equilibrated with 50 ml of phosphate buffered saline (PBS) pH 7.3. Approximately 80 ml of human sera were pooled, centrifuged for 10 minutes at 6000 g, decanted, and diluted 1:5 in PBS and filtered through a 0.22 μm bottle top filter before loading to the sepharose column at 0.5 ml/min at 4° C. The column was then washed with 100 ml PBS and antibodies were eluted with 0.1M glycine, 0.5M NaCl, pH 3.4 to 2.9 at 0.8 ml/min at room temperature. 2 ml fractions were collected and neutralised immediately after elution with 100 μl of 1M Tris-HCl, pH 8.5. Fractions containing antibody were pooled and dialysed twice against 2 litres of PBS. Purified antibodies were concentrated using Centricon YM-10 centrifugal filter units (Millipore, Billerica, Mass.). Antibodies were filtered through a 0.22 μm syringe filter, aliquoted and stored at −80° C. Concentration of purified antibodies was determined with a bicinchoninic acid (BCA) protein assay (Pierce, Rockford, Ill.) using a bovine serum albumin (BSA) standard.

Hybridoma supernatants were filtered through a 0.22 μm bottle top filter and purified on a HiTrap Protein G column (Amersham Biosciences, Piscataway, N.J.) according to manufacturer's protocol. Eluted fractions were processed as described above. The titre of purified antibodies against the corresponding peptide was determined by a standard ELISA as described above.

Plasmodium falciparum In Vitro Cultures

Plasmodium falciparum strains 3D7 and K1 were grown in human O+ red blood cells (Blutspendezentrum SRK beider Basel, Basel, Switzerland) at 5% haematocrit in RPM1 1640 supplemented with Albumax (GibcoBRL, Invitrogen, Carlsbad, Calif.) to a final concentration of 0.5%, and gassed with 4% CO₂ and 3% O₂ in N₂ at 37° C. as described previously (Trager and Jensen). Parasite cultures were synchronised by two sorbitol treatments 10 hours apart (Lambros and Vanderberg).

### Immunofluorescence Assay (IFA)

IFAS were performed on Plasmodium falciparum cultures containing at least 5% late schizonts. Blood smears from strains 3D7 or K1 were fixed with acetone/methanol (1:1). Slides were blocked for 30 minutes with 3% BSA in PBS. Primary antibody reactions were carried out for 1 hour at room temperature in 3% BSA/PBS. Secondary antibodies were applied after 5 washes with PBS and incubated for 1 hour at room temperature in 3% BSA/PBS. Cy3-conjugated goat anti-human IgG (H+ L) (Jackson immunoresearch, West Grove, Pa.) and Cy3-conjugated goat anti-mouse IgGγ (Jackson Immunoresearch) were used at a dilution of 1:500. Slides were washed 5 times with PBS and mounted with Vectashield mounting medium H-100 (Vector Laboratories, Burlingame, Calif., USA) containing DAPI at a concentration of 1 μg/ml. Fluorescense images were captured using a Leitz Dialux 20 fluorescence microscope and a Leica DC 200 digital camera (Leica Microsystems AG, Germany). Images were processed using Adobe PhotoshopCS.

### Analysis of Protection

We analysed the association with protection of the anti-MSP2 antibodies using sera in sample set IV. Protection against clinical malaria was measured by the time delay to first reported clinical episode after the baseline sero-immunological survey (defined as a fever episode with P. falciparum density >20,000 parasite/pt). The relationship of protection with antibody titres was analysed using Kaplan-Meier survival techniques. Age-adjusted and unadjusted log rank chi-square tests were calculated using SAS (version 9, SAS Institute Inc., Cary, N.C., USA,).

### Antibody-Dependent Cellular Inhibition (ADCI) Assay

Monocytes (MN) from healthy, non-malaria-exposed donors were prepared as described earlier [Bouharoun-Tayoun et al]. To wells containing 2×10⁵ MN, 50 μl of an asynchronous parasite culture (P. falciparum strain 3D7 or FC27, respectively) was added at 0.5% parasitemia and 4% hematocrit. Wells were then supplemented with test or control antibody and the total volume adjusted to 100 μl with culture medium. All test and control wells were done in duplicate. After 48 h and 72 h, 50 μl of culture medium was added to each well and after 96 h the ADCI assay was stopped and the final parasitemia was determined by light microscopy on Giemsa stained smears by counting 50,000 red blood cells. For each antibody (Ab) tested, duplicate wells included the following controls 1) non-specific monocytic inhibition, both MN+parasites and MN+N-IgG+parasites. MN with non-specific anti-parasite effect (i.e. without Abs)>15% were excluded from ADCI assays. 2) direct inhibition by control or test IgG, both N-IgG+parasites, and test Abs+parasites. As a positive control we used PIAG, which is IgG purified from a pool of hyperimmune African adults previously found to confer passive protection when transferred to nonimmune individuals. Negative control IgG (N-IgG) was purified from a pool of 1000 French donors with no history of malaria. PIAG and N-IgG were used at a final concentration of 1 mg/ml. Immunopurified test antibodies were used at 15 μg/ml. The specific growth inhibitory index (SGI) which considers the parasite growth inhibition due to the effect of test antibody cooperating with MN was calculated as follows: SGI=100×[1−(% parasitemia with MN and test Abs/% parasitemia test Abs)/(% parasitemia with MN and N—IgG/% parasitemia N-IgG)]. For each tested Ab, SGI were normalized with our internal positive control (PIAG) in order to allow comparison between assays.

### Example 2

### Results

### Design of Long Synthetic MSP2 Peptides and Their Antigenicity.

Two long synthetic MSP2 peptides corresponding to the dimorphic region of 3D7 and FC27 were synthesized and evaluated. The peptide sequences are shown in FIG. 1. Peptide MR141 (3D7-MSP2) includes 88 amino acids of the non-repetitive semi-conserved part of the 3D7 molecule and 40 amino acids of the C-terminal conserved part. Peptide MR144A (FC27-MSP2) represents the other allelic family (45 amino acids of the non-repetitive dimorphic part) plus the 40 amino acids of the C-terminal conserved part. An additional peptide covering the 40 amino acids of the C-terminal conserved part (MR140) was also synthesized. The two C-terminal cysteine residues were reduced in all peptides.

The antigenicity of the synthetic peptides was evaluated by IgG ELISA using three sets of human sera from different age groups and different malaria endemic areas. The peptides MR141 and MR144A included the family-specific part of 3D7 or FC27 MSP2. The prevalence of antibody against both peptides was high; 96% of the tested adult sera from Papua New Guinea (n=80) recognized the 3D7 peptide MR141 and 93% recognized the FC27 peptide MR144A, and 43% recognized peptide MR140 that represents the conserved C-terminus. The median OD value was 0.84 for the peptide representing the 3D7 family (quartiles: 0.31; 1.83). The median OD value for the FC27 peptide was 0.42 (quartiles: 0.09; 0.49) and 0.16 (0.11; 0.27) for MR140. Dilution of sera was 1:1000 for assays on peptides MR140 and MR144A and 1:400 for assays on MR140.

Peptide recognition by sera of 6 to 14 months old Tanzanian children was also assessed. The ELISA results obtained were compared to those previously obtained in the same sera but using recombinant proteins as antigens (Irion, Beck, and Smith). These antigens were expressed in E. coli and corresponded to the two MSP2 family-specific domains (3D7 and FC27) or to a fusion of the conserved N- and C-termini. The prevalence of sera with IgG reactivity to the synthetic peptides agreed well with the positivity obtained for the recombinant proteins (Table 1). This indicates similar antigenic properties of the synthetic peptides and the recombinant proteins. Compared to the results obtained in adults, positivity against the dimorphic parts was higher in adults than in young children, but positivity against the conserved C-terminal part was higher in children, which is a surprising finding.

TABLE 1
Comparison of prevalence of antibodies to synthetic MSP2 peptides and recombinant MSP2 domains in
Tanzanian infant sera.

| | | | Classical approach used to | | |
|---|---|---|---|---|---|
| | Data description | | define positivity¹⁾ | | |
| | Mean OD in | | Cutoffs | Proportion | Latent class model |
| | negative | Mean OD in | (units) mean | above cutoff | (% positive, 95% |
| Antigen | controls (S.D.) | test sera (S.D.) | OD + 2 S.D. | (positivity) | confidence levels)²⁾ |
| MR141 (3D7 fsp & | 0.002 (0.004) | 0.221 (0.293) | 0.011 | 0.728 | 0.73 (0.62, 0.83) |
| cons) | | | | | |
| recombinant 3D7 | 0.005 (0.005) | 0.05 (0.08) | 0.015 | 0.525 | 0.75 (0.65, 0.84)³⁾ |
| fsp | | | | | |
| MR144A (FC27 | 0.003 (0.005) | 0.214 (0.353) | 0.012 | 0.772 | 0.82 (0.72, 0.90) |
| fsp & cons) | | | | | |
| recombinant FC27 | 0.090 (0.095) | 0.30 (0.24) | 0.280 | 0.443 | 0.78 (0.64, 0.89)³⁾ |
| fsp | | | | | |
| MR140 (cons) | 0.009 (0.009) | 0.566 (0.707) | 0.028 | 0.781 | 0.82 (0.72, 0.91) |
| rec. N-C-conserved | 0.281 (0.284) | 2.07 (3.07) | 0.848 | 0.540 | 0.84 (0.78, 0.91)³⁾ |

¹⁾Determination of proportion of positive sera by classical approach (cutoff = OD of control sera + 2 * standard deviation (S.D.))
²⁾Proportion of positive sera according to latent class model (Irion et al., 2002)
³⁾Data taken from Irion et al. (2002)
⁴⁾This peptide encompasses the non-repetitive family-specific part of 3D7-MSP2

Association of Peptide-Specific Antibody with Protection

In the Kaplan-Meier analysis of incidence of clinical episodes in relation to optical densities (Table 2), the strongest protective effect was found for IgG3 antibodies against the 3D7 peptide however, when adjusting for age, the statistical significance of this effect was borderline significant. Anti-MSP2 antibodies in baseline sera were positively correlated with age (Spearman correlation, Table 3). This led to substantial decline of the protective effect after correction for age. Also for the FC27 peptide, the strongest, but borderline significant, protective effect was seen for IgG3 antibodies. For the C-terminal constant domain no protective effect at all was observed.

TABLE 2
OD distributions and incidence of morbidity by OD

| | | Mean OD by | | | | Incidence of | | | |
|---|---|---|---|---|---|---|---|---|---|
| | | quartile of the OD | | | | morbidity by quartile | | | |
| | | distribution | | | | of the OD distribution¹⁾ | | | |
| Antigen | Isotype | 1st | 2nd | 3rd | 4th | 1st | 2nd | 3rd | 4th |
| MR141 | IgG1 | 0.090 | 0.112 | 0.147 | 0.342 | 3.38 | 2.10 | 1.59 | 1.84 |
| | IgG3 | 0.093 | 0.122 | 0.181 | 0.383 | 4.93 | 2.15 | 1.35 | 1.69 |
| | IgG | 0.127 | 0.260 | 0.698 | 1.609 | 3.28 | 2.26 | 1.82 | 1.52 |
| MR144A | IgG1 | 0.103 | 0.129 | 0.161 | 0.267 | 2.43 | 2.13 | 2.41 | 1.56 |
| | IgG3 | 0.160 | 0.200 | 0.239 | 0.365 | 2.86 | 2.08 | 2.29 | 1.46 |
| | IgG | 0.099 | 0.128 | 0.178 | 0.620 | 2.29 | 2.80 | 1.86 | 1.64 |

¹⁾Incidence density of first episodes of clinical malaria (fever & parasitaemia) following the survey when the blood sample was collected.

TABLE 3
Relationships between ODs, morbidity and age

| | | | Chi-squared | | | |
|---|---|---|---|---|---|---|
| | | | statistics | | P-values | |
| | | Age | Unad- | | Unad- | |
| Antigen | Isotype | corr.¹⁾ | justed²⁾ | Adjusted³⁾ | justed²⁾ | Adjusted³⁾ |
| MR141 | IgG1 | 0.23 | 4.37 | 0.98 | 0.04 | 0.3 |
| | IgG3 | 0.19 | 8.97 | 3.79 | 0.003 | 0.052 |
| | IgG | 0.16 | 5.75 | 2.74 | 0.02 | 0.098 |
| MR144A | IgG1 | 0.18 | 2.52 | 0.48 | 0.11 | 0.5 |
| | IgG3 | 0.14 | 3.98 | 1.47 | 0.046 | 0.2 |
| | IgG | 0.21 | 1.98 | 0.28 | 0.2 | 0.6 |

¹⁾Spearman correlation with age.
²⁾Log rank chi-square from Kaplan-Meier analysis of the incidence of morbidity in relation to OD, unadjusted for age.
³⁾Log rank chi-square from age adjusted Kaplan-Meier analysis.

Immunogenicity of peptides representing the two allelic MSP2 families. We immunized mice with MR141 (3D7-MSP2) and MR144A (FC27-MSP2) to determine immunogenicity of the peptides. CB6F1 mice injected 3 times with 20 μg of peptide with Montanide consistently gave antibody titres of 4×10⁵. The C-terminal conserved part of MSP2 contained in peptides MR141 and MR144A also proved to be immunogenic giving titres ranging from 5×10⁴ to 4×10³. see Tables 4a & b

TABLE 4a

| | | | ELISA | | | | |
|---|---|---|---|---|---|---|---|
| Mouse | | | Antigenic | GMT | SD | Responders | |
| strain | Antigens | Adjuvants | peptide | (Log10) | (Log10) | (titer > 1000)^{a} | IFAT |
| A/J | Mix | Alum | 3D7-1 * | 3.99 | 1.53 | 3/4 | Positive |
| | MSP2 | | FC27-1° | 4.61 | 1.19 | 3/4 | |
| | | | 3D7-2″ | 4.57 | 1.42 | 3/4 | |
| | | | C-region^{†} | 4.57 | 1.42 | 3/4 | |

| | Rec prot 3D7 | 5.34 | done on pool | | |
|---|---|---|---|---|---|
| | Rec prot FC27 | 5.34 | done on pool | | |
| GLA-SE | 3D7-1 * | 4.56 | 0.83 | 4/4 | Positive |
| | FC27-1° | 5.46 | 0.24 | 4/4 | |
| | 3D7-2″ | 5.44 | 0.60 | 4/4 | |
| | C-region^{†} | 5.44 | 0.00 | 4/4 | |

| | | |
|---|---|---|
| Rec prot 3D7 | 5.34 | done on pool |
| Rec prot FC27 | 5.34 | done on pool |

TABLE 4b

| | | | ELISA | | | | |
|---|---|---|---|---|---|---|---|
| Mouse | | | Antigenic | GMT | SD | Responders | |
| strain | Antigens | Adjuvants | peptide | (Log10) | (Log10) | (titer > 1000)^{a} | IFAT |
| C3H | Mix | Alum | 3D7-1* | 4.85 | 0.39 | 4/4 | Positive |
| | MSP2 | | FC27-1° | 2.43 | 1.19 | 1/4 | |
| | | | 3D7-2″ | 4.85 | 0.48 | 4/4 | |
| | | | C-region^{†} | 2.60 | 1.38 | 1/4 | |

| | Rec prot 3D7 | 4.86 | done on pool | | |
|---|---|---|---|---|---|
| | Rec prot FC27 | 3.91 | done on pool | | |
| GLA-SE | 3D7-1* | 4.86 | 0.00 | 4/4 | Positive |
| | FC27-1° | 5.18 | 0.28 | 4/4 | |

| | | |
|---|---|---|
| 3D7-2″ | 4.48 | done on pool |
| C-region^{†} | 4.48 | done on pool |
| Rec prot 3D7 | 5.43 | done on pool |
| Rec prot FC27 | 5.91 | done on pool |
| Rec prot FC27 | 2.95 | done on pool |

*correspond to MSP2-3D7 111-206 (LR186)
°correspond to MSP2-FC27 120-207 (LR200Ao)
″correspond to MSP2-3D7 111-238 (MR141)
^{†}correspond to constant region MSP2-3D7/FC27 198--238 (MR140; 3D7 numbering)
^{a}correspond to the last dilution above mean OD value + 3SD of the naive mice sera

Monoclonal antibodies raised against synthetic peptide MR141 recognize parasite-derived MSP2. Monoclonal antibodies were raised against peptide MR141 (3D7-MSP2). 24 hybridoma cultures were positive against the peptide used for immunization (MR141). Five of the 24 positive culture supernatants were also positive against peptide MR140, suggesting that they recognize an epitope in the conserved C-terminal part of MSP2. Eleven of 24 of the ELISA-positive cultures produced antibody that reacted with the merozoite surface of 3D7 parasites in indirect immunofluorescence assays (IFA). Four IFA-positive cultures were chosen for cloning. IgG subclasses IgG1, IgG2a, and IgG2b were found among the hybridoma clones. FIG. 4C shows an immunofluorescence image with monoclonal antibody from a clone recognizing an epitope in the 3D7 family-specific domain. FIG. 4D shows immunofluorescence reactivity of antibody from another clone, recognizing an epitope in the C-terminal conserved domain. Both monoclonal antibodies gave a pattern characteristic for surface staining in mature schizonts. The production of hybridomas from mice immunized with MR144A failed twice for unknown reasons.

Synthetic peptide MR140 was recognized by a monoclonal antibody that had been raised against a recombinant fusion of the two conserved terminal regions (Irion). This corroborates the statement that structural differences between the recombinant protein and the synthetic peptide are limited and do not lead to differential recognition by antibodies.

### Specificity and Distribution of IgG Subclasses of Human Antibodies Purified on Synthetic MSP2 Peptides.

Sera from Papua New Guinean adults that gave high OD values (>1) in ELISA to MR141 (3D7-MSP2) or MR144A (FC27-MSP2) were pooled for affinity purification of antibodies on the corresponding peptides. The affinity purification yielded 2 mg of anti-MR141 and 0.8 mg of anti-MR144A antibody. The antibodies purified on peptide MR141 represented ˜ 1/700 of total IgG and corresponded to a serum concentration of 21 μg/ml. The antibody purified on peptide MR144A represented ˜ 1/800 of total IgG and a serum concentration of 18 μg/ml. The reactivity and specificity of purified antibodies was confirmed by ELISA (FIG. 4). A weak cross-reactivity to the peptides representing the alternative MSP2 family was found. As shown in FIG. 4 this reactivity can be fully attributed to the conserved C-terminal part common to both peptides (see FIG. 1).

Affinity-purified antibodies were used in immunofluorescence assays. The antibody staining obtained was typical for a merozoite surface protein (FIG. 3A, B). Thus, naturally occurring antibodies reactive to our synthetic peptides also recognize native parasite-derived MSP2. This indicates that the antigenic properties of our peptides are comparable to those of native MSP2.

IgG subclasses of the peptide-purified human MSP2 antibodies were determined and IgG3 was found to be the dominant subclass in antibody preparations (FIG. 5).

### Immunogenicity of the Peptides in Mice

Applicants immunized mice with an antigenic cocktail of LR186 (3D7-MSP2, SEQ ID No 2) and MR144A (FC27-MSP2; SEQ ID No 3) to determine immunogenicity of the peptides. CB6F1 mice injected 3 times with 20 μg of peptide with Montanide consistently gave antibody titres of 4×10⁵. The C-terminal conserved part of MSP2 contained in peptides LR186 and MR144A also proved to be immunogenic, giving titers ranging from 5×10⁴ to 4×10⁵. Both peptides induced antibodies that reacted with parasites in IFA, with titers of at least 1/2500. The induced antibodies also recognize the protein in Western blot.

Monoclonal antibodies raised against the synthetic peptide LR186 (3D7-MSP2) recognized parasite-derived MSP2, and gave a pattern characteristic for surface staining in mature schizonts when tested in an IFA on parasite-infected erythrocytes. They also recognized the protein in Western blot. On the basis of these results, LR186 (MSP2-3D7) and MR144A (MSP2—FC27) were also selected for further development as potential vaccine candidates.

### In Vitro Assays for the Assessment of the Inhibitory Potential of Peptide-Purified MSP2 Antibodies.

Applicants tested the inhibitory potential of the affinity-purified IgG antibodies in a direct growth inhibition assay and in cooperation with human monocytes in an antibody-dependent cellular inhibition (ADCI) assay. In the direct growth inhibition assay neither the affinity-purified human antibodies nor the tested mouse monoclonal antibodies inhibited parasite growth at the tested concentrations (160 μg/ml or 250 μg/ml, respectively; data not shown). In cooperation with monocytes the human antibody purified on peptide MR144A inhibited both, in vitro growth of strain K1 (FC27-type MSP2), and 3D7 (3D7-type MSP2). All results are mean values±S.D. of two independent experiments. The specific growth inhibition was 54% (±6%) on strain K1 and 113% (±3%) on strain 3D7. For the 3D7 construct Applicants used an antibody purified on peptide LR186, which is a shorter version of MR141, only comprising the family-specific part of 3D7 MSP2 and 8 amino acids of the conserved C-terminal part. These specific antibodies also inhibited both strains in the ADCI assay (103% (±4%) and 52% (±3%) on 3D7 and K1 strain, respectively).

1. An antigenic peptide derived from the constant part of at least one of the two allelic families of the merozoite surface protein 2 (MSP2) of Plasmodium falciparum characterized in that said antigenic peptide is selected from the group comprising the amino acid sequences
SEQ ID No 1:
AEASTSTSSENPNHKNAETNPKGKGEVQEPNQANKETQNNSNVQQDSQTKSNVPPTQDADTKSPTAQPEQAEN SAPTAEQTESPELQSAPENKGTGQHGHMHGSRNNHPQNTSDSQKECTDGNKENCG,
SEQ ID No 2:
AEASTSTSSENPNHKNAETNPKGKGEVQEPNQANKETQNNSNVQQDSQTKSNVPPTQDADTKSPTAQPEQAEN SAPTAEQTESPELQSAPENKGTG,
SEQ ID No 3:
ESSSSGNAPNKTDGKGEESEKQNELNESTEEGPKAPQEPQTAENENPAAPENKGTGQHGHMHGSRNNHPQNTS DSQKECTDGNKENCG,
biologically active fragments thereof, molecular chimeras thereof, combinations thereof and/or variants thereof.

2. The antigenic peptide of claim 1, characterized in that a biologically active fragment thereof is selected from the group comprising the amino acid sequences
SEQ ID N^{o} 4:
ESSSSGNAPNKTDGKGEESEKQNELNESTEEGPKAPQEPQTAENENPA,
SEQ ID N^{o} 5:
APENKGTGQHGHMHGSRNNHPQNTSDSQKECTDGNKENCG,
SEQ ID N^{o} 6:
AEASTSTSSENPNHKNAETNPKGKGEVQEPNQANKETQNNSNVQQDSQTK
SNVPPTQDADTKSPTAQPEQAENSAPTAEQTESPELQS,
and
SEQ ID N^{o} 7:
APENKGTG.

3. An antigenic cocktail composition derived from the constant part of at least one of the two allelic families of the merozoite surface protein 2 (MSP2) of Plasmodium falciparum comprising at least 2 antigenic peptides according to claim 1.

4. The antigenic cocktail composition of claim 3 comprising the antigenic peptide of SEQ ID No 2 and the antigenic peptide of SEQ ID No 3.

5. The antigenic cocktail composition of claim 3, characterized in that said antigenic peptides are linked.

6. The antigenic cocktail composition of claim 3 characterized in that said antigenic peptides are linked by a peptide bond or by the way of a linker.

7. The antigenic cocktail composition of claim 6 characterized in that said linker is Poly ethylene glycol (PEG).

8. An antibody characterized in that it recognizes an antigenic peptide of claim 1 or an antigenic cocktail composition of claim 3.

9. The antibody of claim 8 characterized in that it selected from the group comprising the IgG1, IgG2, IgG2a, IgG2b, IgG3 and IgG4.

10. The antibody of claim 9 characterized in that it is IgG3.

11. A vaccine composition useful to stimulate an immune response in a mammal characterized in that it comprises the antigenic peptide of claim 1, an antigenic cocktail composition of claim 3 or an antibody of claim 8.

12. The vaccine composition of claim 11 further comprising an adjuvant.

13. A purified and isolated nucleic acid sequence comprising
i) a nucleotide sequence encoding an antigenic peptide of claim 1,
ii) a nucleic acid sequence complementary to i),
iii) a degenerated nucleic acid sequence of i) or ii),
iv) a nucleic acid sequence capable of hybridizing under stringent conditions to i), ii) or iii),
v) a nucleic acid sequence encoding a truncation or an analog of the antigenic peptide of claim 1,
vi) and/or a fragment of i), ii), iii), iv) or v) encoding a biologically active fragment of ef said antigenic peptide of claim 1.

14. The purified and isolated nucleic acid sequence of claim 13 comprised in an expression vector.

15. The purified and isolated nucleic acid sequence of claim 13 comprised in a host cell.

16. Use of the vaccine composition of claim 11, in the manufacture of a medicament for the treatment and/or prevention of malaria.

17. A method for determining the presence of an antigenic peptide of claim 1 or an antigenic cocktail composition of claim 3 in a sample comprising:
i) contacting the sample with an antibody directed to the antigenic peptide of claim 1 or to the antigenic cocktail composition of claim 3, and
ii) determining whether said antibody binds to a component of said sample.

18. A method for determining the presence of antibodies to a peptide of claim 1 or an antigenic cocktail composition of 3 in a sample comprising:
i) contacting said sample with the antigenic peptide of claim 1 or the antigenic cocktail composition of claim 3, and
ii) determining whether antibodies bind to a component of said antigenic peptide of claim 1 or to said antigenic cocktail composition of claim 3.

19. A protein characterized in that it comprises at least one antigenic peptide of claim 1 or an antigenic cocktail composition of claim 3.

20. A kit comprising the vaccine composition of claim 11, optionally with reagents and/or instructions for use.

21. A kit for the in vitro diagnosis of malaria in an individual likely to be infected by a plasmodium falciparum which contains: an antigenic peptide derived from the constant part of at least one of the two allelic families of the merozoite surface protein 2 (MSP2) of Plasmodium falciparum according to claim 1 or an antigenic cocktail composition of claim 3, the reagents for the constitution of the medium appropriate for carrying out the antigen-antibody reaction, the reagents making possible the detection of the complex formed.

22. An A hybridoma expressing an antibody according to claim 8.

23. The expression vector of claim 14 comprised in a host cell.


SEQUENCE LISTING (7 sequences)

SEQ ID NO: 1 - protein, 128 aa, Artificial
  synthetic peptide
AEASTSTSSENPNHKNAETNPKGKGEVQEPNQANKETQNNSNVQQDSQTKSNVPPTQDADTKSPTAQPEQAENSAPTAEQTESPELQSAPENKGTGQHGHMHGSRNNHPQNTSDSQKECTDGNKENCG

SEQ ID NO: 2 - protein, 96 aa, Artificial
  synthetic peptide
AEASTSTSSENPNHKNAETNPKGKGEVQEPNQANKETQNNSNVQQDSQTKSNVPPTQDADTKSPTAQPEQAENSAPTAEQTESPELQSAPENKGTG

SEQ ID NO: 3 - protein, 88 aa, Artificial
  synthetic peptide
ESSSSGNAPNKTDGKGEESEKQNELNESTEEGPKAPQEPQTAENENPAAPENKGTGQHGHMHGSRNNHPQNTSDSQKECTDGNKENCG

SEQ ID NO: 4 - protein, 48 aa, Artificial
  synthetic peptide
ESSSSGNAPNKTDGKGEESEKQNELNESTEEGPKAPQEPQTAENENPA

SEQ ID NO: 5 - protein, 40 aa, Artificial
  synthetic peptide
APENKGTGQHGHMHGSRNNHPQNTSDSQKECTDGNKENCG

SEQ ID NO: 6 - protein, 88 aa, Artificial
  synthetic peptide
AEASTSTSSENPNHKNAETNPKGKGEVQEPNQANKETQNNSNVQQDSQTKSNVPPTQDADTKSPTAQPEQAENSAPTAEQTESPELQS

SEQ ID NO: 7 - protein, 8 aa, Artificial
  synthetic peptide
APENKGTG